(19) United States
(12) Patent Application Publication
(10) Pub. No.: US 2007/0083480 A1
(43) Pub. Date: Apr. 12, 2007

(54) Operation information analysis device and method for analyzing operation information

(75) Inventors: Takashi Ozaki, Tokyo (JP); Koichi Tashiro, Sagamihara-shi (JP); Chie Imamiya, Tokyo (JP); Akinobu Uchikubo, Iruma-shi (JP); Masakazu Gotanda, Sagamihara-shi (JP); Takeaki Nakamura, Tokyo (JP)

Correspondence Address: Thomas Spinelli;Scully, Scott, Murphy & Presser, Suite 300, 400 Garden City Plaza, Garden City, NY, 11530, US

(73) Assignee: Olympus Medical Systems Corp., Tokyo (JP)

(21) Appl. No.: 11/520,159

(22) Filed: Sep. 13, 2006

(30) Foreign Application Priority Data
Sep. 16, 2005 (JP) 2005-270702
Nov. 8, 2005 (JP) 2005-324010

(51) Int. Cl. A61B 5/11

(52) U.S. Cl. 706/924

(57) ABSTRACT

An operation information analysis device according to the present invention includes: a medical information input section for inputting various kinds of medical information during an operation from a system controller; a data storage section for adding time information such as a time stamp to the various kinds of medical information during the operation, and for constructing databases specified for each medical procedure by making the various kinds of medical information into files for each kind of medical information and storing the databases; a data analysis section for analyzing the medical information which is stored in the data storage section and which is formed into the databases, for each medical procedure; an analysis condition input section for inputting various information to the data analysis section; and a display device for displaying results of analysis performed by the data analysis section.

This application claims benefit of Japanese Application No. 2005-270702 filed on Sep. 16, 2005, and No. 2005-324010 filed on Nov. 8, 2005, the contents of which are incorporated by this reference.

### BACKGROUND OF THE INVENTION

1. Field of the Invention

The present invention relates to an operation information analysis device which analyzes information acquired from plural medical instruments during an operation, and a method for analyzing the operation information.

2. Description of the Related Art

In recent years, the endoscopic operation system which enables medical procedure to be performed by the use of an endoscope, has been prevalent and used for various kinds of medical instruments.

The medical instruments used in such endoscopic operation system include an electrocautery device, an ultrasonic device, a pneumoperitoneum device, and the like, in addition to the electronic endoscope system. For example, as proposed in Japanese Patent Laid-Open No. 2003-76786 or Japanese Patent Laid-Open No. 2003-70746, these devices are integrally managed as a system and controlled by an operation device arranged under a system controller.

Further, in recent years, along with the development of medical technology, the kinds of medical instruments have been increased and their functions tend to be enhanced. As the medical instruments, various kinds of devices, such as an electrocautery device, ultrasonic treatment device and laser scalpel are prepared. These medical instruments may be used singly, and may also be used as a composite medical system.

In such medical system, for example, as proposed in Japanese Patent Laid-Open No. 2002-233535, there is an operation system in which various kinds of medical instruments are controlled in a centralized manner by a system controller. Such operation system performs monitoring and recording of output state data outputted from various medical instruments during an operation, that is, the output state data such as, for example, operation instrument data such as electrocautery output data obtained from an electrocautery device, and biomedical data such as of blood pressure, pulse and blood transfusion quantity of a patient, which are obtained from a patient monitoring device. The operator reads and analyzes the plural kinds of stored output state data after the operation, so as to prepare a medical record or the like, and also to prepare a scientific essay to be presented at a meeting, or the like.

### SUMMARY OF THE INVENTION

An operation information analysis device according to one aspect of the present invention comprises: an information input section for inputting plural pieces of instrument information of plural medical instruments and plural pieces of biomedical information; an information storage section for adding time information to the plural pieces of instrument information and the plural pieces of biomedical information, that are inputted by the information input section, and for classifying and storing the plural pieces of instrument information and the plural pieces of biomedical information in association with predetermined codes, as information to be analyzed; a comparison information input section for inputting comparison information to be compared with the information to be analyzed; and an information analysis section for analyzing the information to be analyzed by comparing the information to be analyzed with the comparison information.

An operation information analysis method according to one aspect of the present invention comprises: inputting plural pieces of instrument information of plural medical instruments and plural pieces of biomedical information; adding time information to the inputted plural pieces of instrument information and the inputted plural pieces of biomedical information, and classifying and storing the plural pieces of instrument information and the plural pieces of biomedical information in association with predetermined codes, as information to be analyzed; and analyzing the information to be analyzed by comparing the information to be analyzed with comparison information to be compared with the information to be analyzed.

An operation information analysis device according to one aspect of the present invention comprises: a data recording section for recording output state data including operation instrument data outputted from medical instruments, in association with identification mark data for identifying each treatment performed by the medical instruments, the identification mark data being inputted from a bookmark input section; and a display processing section for comparably displaying first output state data representing the output state data and the identification mark data that are recorded in the data recording section, on a same time base, and second output state data representing other output state data and other identification mark data that are recorded beforehand in the data recording section, on the same time base.

An operation information analysis method according to one aspect of the present invention comprises: recording output state data including operation instrument data outputted from medical instruments, in association with identification mark data for identifying each treatment performed by the medical instruments, the identification mark data being inputted from a bookmark input section; and comparably displaying first output state data representing the output state data and the identification mark data that are recorded, on a same time base, and second output state data representing other output state data and other identification mark data that are recorded beforehand in the data recording section, on the same time base.

### BRIEF DESCRIPTION OF THE DRAWINGS

FIG. 1 is a constructional figure showing a constitution of an endoscopic operation system according to a first embodiment of the present invention;

FIG. 2 is a block diagram showing a constitution of an operation information analysis device in FIG. 1;

FIG. 3 is a flow chart showing a process flow in the operation information analysis device shown in FIG. 2;

FIG. 4 is a first figure explaining the processing in FIG. 3;

FIG. 5 is a second figure explaining the processing in FIG. 3;

FIG. 6 is a third figure explaining the processing in FIG. 3;

FIG. 7 is a fourth figure explaining the processing in FIG. 3;

FIG. 8 is a fifth figure explaining the processing in FIG. 3;

FIG. 9 is a sixth figure explaining the processing in FIG. 3;

FIG. 10 is a seventh figure explaining the processing in FIG. 3;

FIG. 11 is an eighth figure explaining the processing in FIG. 3;

FIG. 12 is a figure showing an entire constitution of an endoscopic operation system according to a second embodiment of the present invention;

FIG. 13 is a figure explaining an operation analysis device connected to the endoscopic operation system shown in FIG. 12;

FIG. 14 is a block diagram showing a circuit constitution of the operation analysis device shown in FIG. 13;

FIG. 15 is a flow chart showing a data acquisition recording operation performed by a CPU of the operation analysis device in FIG. 14;

FIG. 16 is a flow chart showing comparison display processing performed by the CPU of the operation analysis device in FIG. 14;

FIG. 17 is a figure showing a first comparison image showing an example of comparison analysis with reference output state data, displayed on the monitor in FIG. 14;

FIG. 18 is a figure showing a second comparison image showing an example of comparison analysis of the skill of a same operator, displayed on the monitor in FIG. 14;

FIG. 19 is a flow chart showing comparison window display processing performed by the CPU of the operation analysis device shown in FIG. 14;

FIG. 20 is a figure showing an output comparison window which is an example of the comparison window displayed on the monitor in FIG. 14; and

FIG. 21 is a figure showing a treatment time comparison window which is an example of the comparison window displayed on the monitor in FIG. 14.

### DETAILED DESCRIPTION OF THE PREFERRED EMBODIMENTS

In the following, embodiments according to the present invention will be described with reference to the accompanying drawings.

### First Embodiment

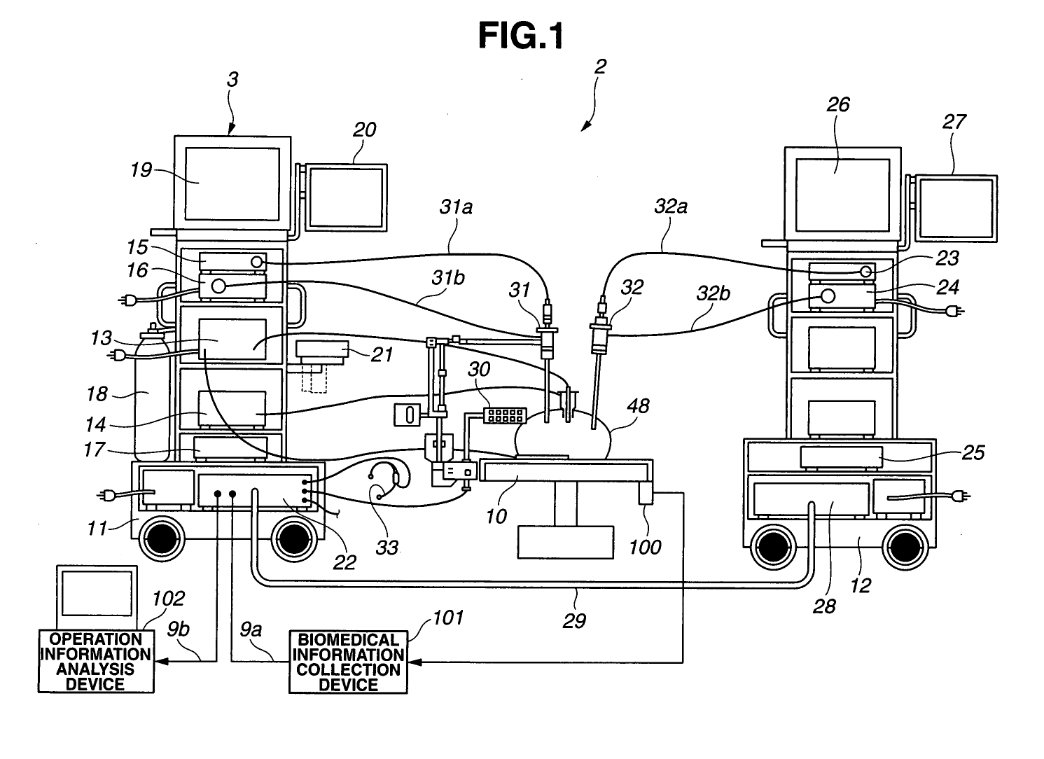

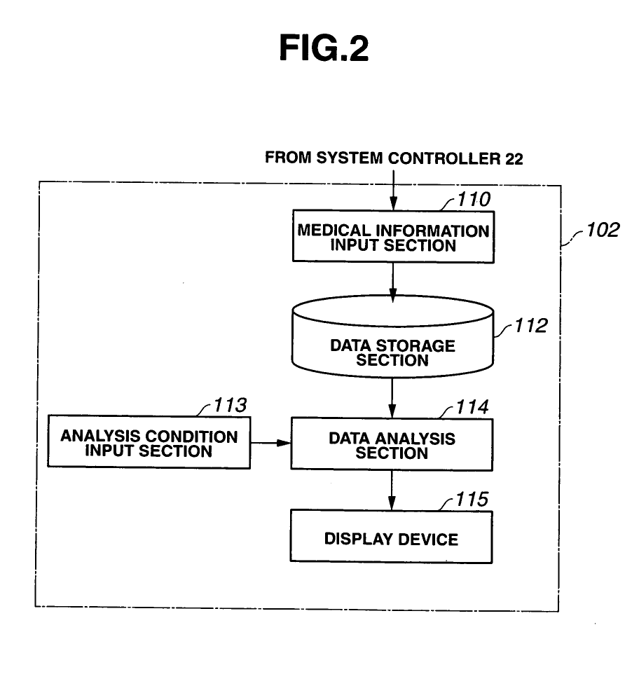

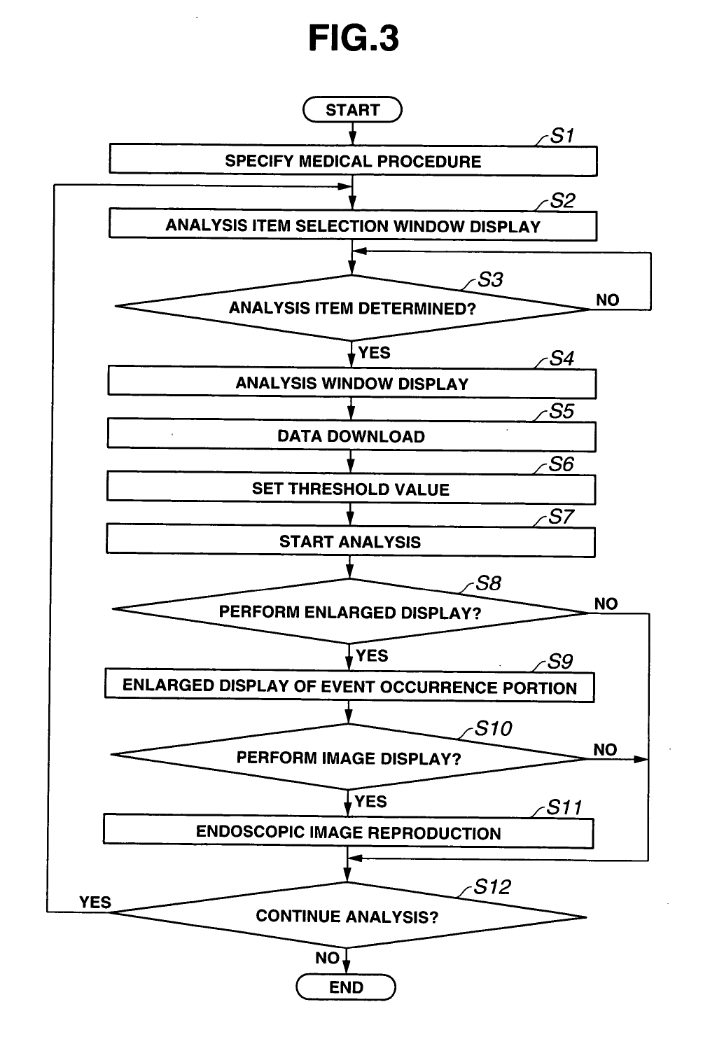

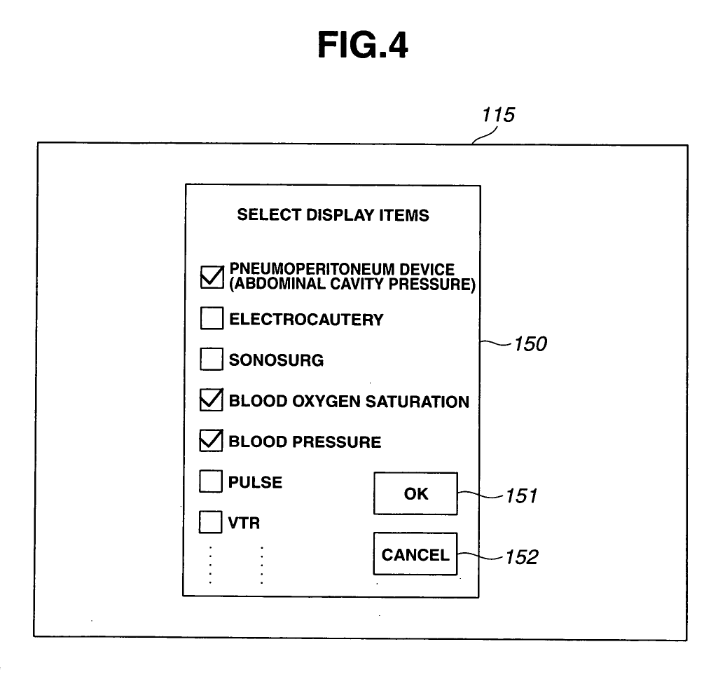

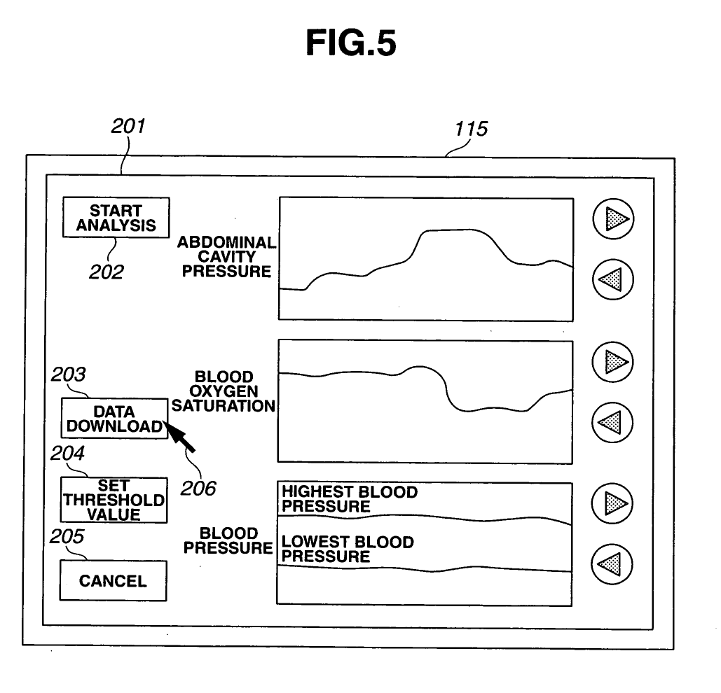

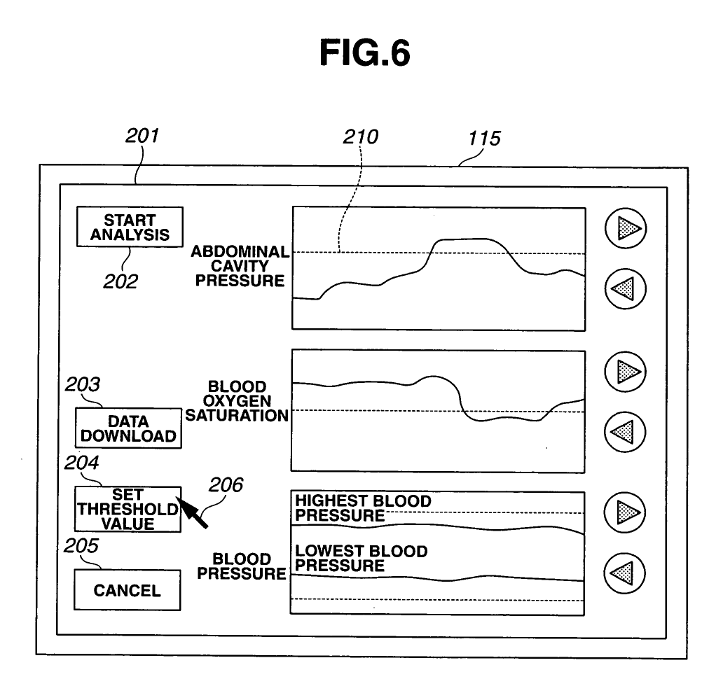

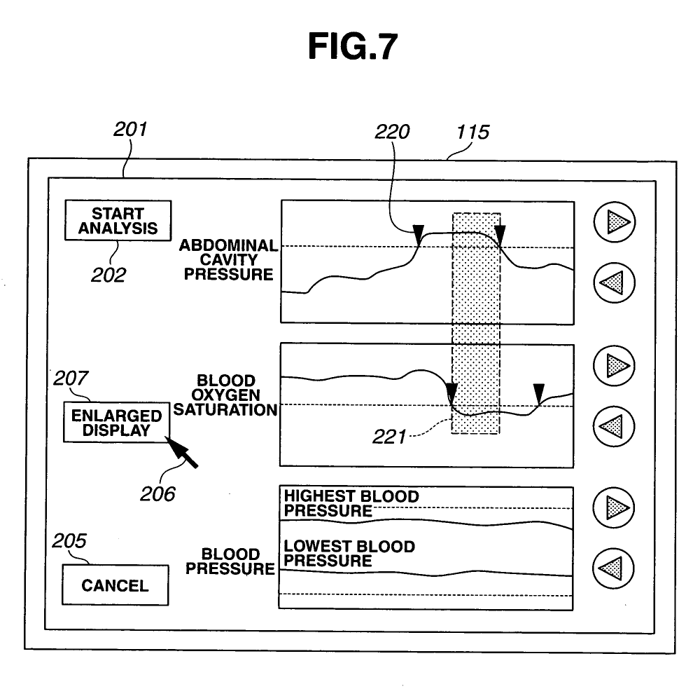

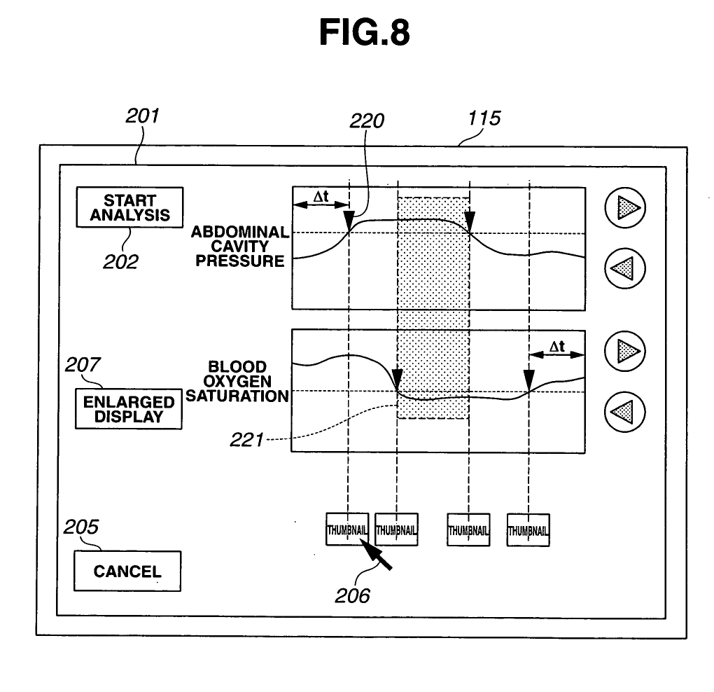

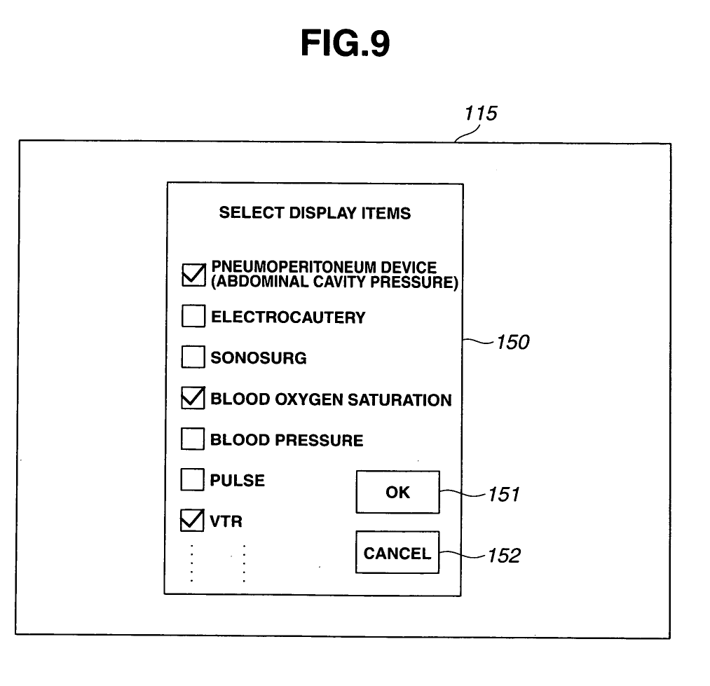

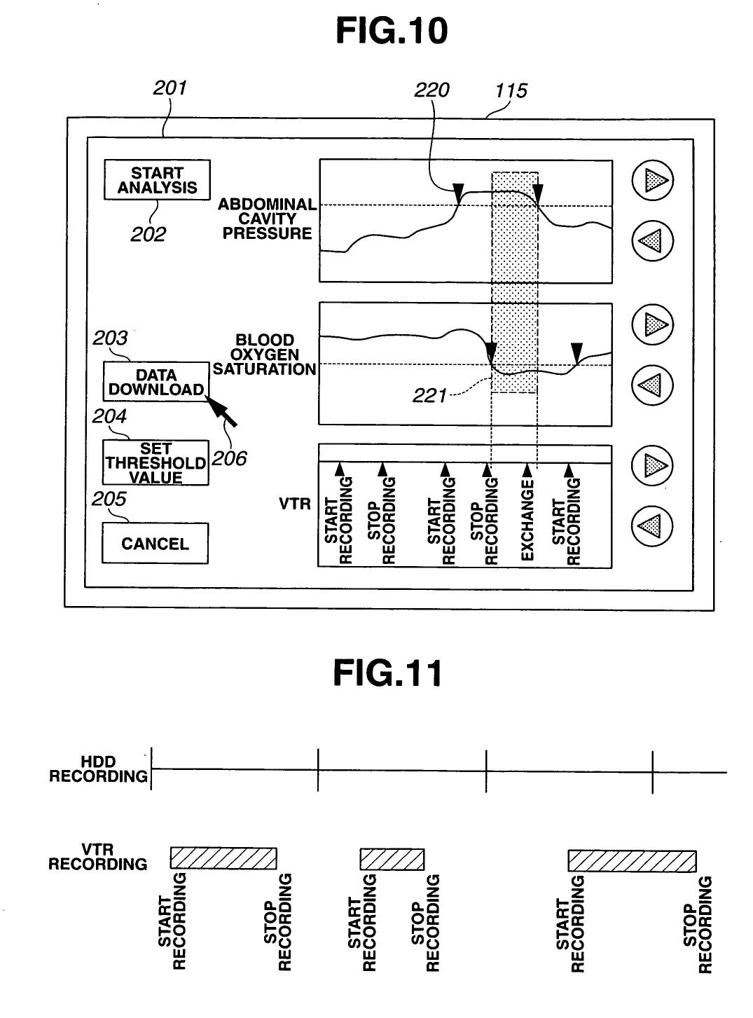

FIG. 1 to FIG. 11 are figures relating to a first embodiment according to the present invention. In the figures, FIG. 1 is a constructional figure showing a constitution of an endoscopic operation system. FIG. 2 is a block diagram showing a constitution of an operation information analysis device in FIG. 1. FIG. 3 is a flow chart showing a process flow in the operation information analysis device in FIG. 2. FIG. 4 is a first figure explaining the processing in FIG. 3. FIG. 5 is a second figure explaining the processing in FIG. 3. FIG. 6 is a third figure explaining the processing in FIG. 3. FIG. 7 is a fourth figure explaining the processing in FIG. 3. FIG. 8 is a fifth figure explaining the processing in FIG. 3. FIG. 9 is a sixth figure explaining the processing in FIG. 3. FIG. 10 is a seventh figure explaining the processing in FIG. 3. FIG. 11 is a eighth figure explaining the processing in FIG. 3.

First, an entire constitution of an endoscopic operation system 3 which is a medical system arranged in an operating room 2 is explained by using FIG. 1.

As shown in FIG. 1, a patient bed 10 on which a patient 48 lies, and an endoscopic operation system 3 are arranged in the operating room 2. This endoscopic operation system 3 has a first cart 11 and a second cart 12.

On the first cart 11, there are mounted as medical instruments which are devices to be controlled, for example, devices such as an electrocautery device 13, pneumoperitoneum device 14, endoscopic camera device 15, light source device 16, videotape recorder (VTR) 17, and a gas cylinder 18 filled with carbon dioxide or the like. The endoscopic camera device 15 is connected to a first endoscope 31 via a camera cable 31a. The light source device 16 is connected to the first endoscope 31 via a light guide cable 31b.

Further, on the first cart 11, there are mounted a display device 19, a first centralized display panel 20 constituted such as, for example, by a liquid crystal display (LCD), and an operation panel 21 which is a first touch panel section, and the like. The display device 19 is, for example, a TV monitor which displays an endoscope image and the like.

The centralized display panel 20 serves as a display section capable of selectively displaying all the data during an operation. The operation panel 21 is constituted, for example, by a display section such as a liquid crystal display and a touch panel integrally provided on the display section, and serves as a centralized operation device which is operated by a nurse or the like located in a non-sterilized area, so as to control each of the medical instruments of the endoscopic operation system 3.

Further, on the first cart 11, there is mounted a system controller 22 which performs control of each of the above described medical instruments arranged on the first cart 11. The system controller 22 is connected with the above described electrocautery device 13, pneumoperitoneum device 14, endoscopic camera device 15, light source device 16, and VTR 17 via a communication line (not shown). The system controller 22 is arranged so as to be connected with a headset type microphone 33. Thereby, the system controller 22 is arranged to be able to recognize the voice inputted from the microphone 33, and to control each instrument in accordance with the voice command of the operator.

The system controller 22 manages the operation information and control information on the above described electrocautery device 13, pneumoperitoneum device 14, endoscopic camera device 15, light source device 16, and VTR 17 in real time, and captures the endoscopic image obtained by the endoscopic camera device 15.

On the other hand, on the second cart 12, there are mounted an endoscopic camera device 23, light source device 24, image processing device 25, display device 26, which are devices to be controlled, and a second centralized display panel 27 constituted such as by a liquid crystal display (LCD).

The endoscopic camera device 23 is connected to a second endoscope 32 via a camera cable 32a. The light source device 24 is connected to the second endoscope 32 via a light guide cable 32b.

The display device 26 displays an endoscopic image and the like captured by the endoscopic camera device 23. The second centralized display panel 27 is arranged to be able to selectively display all the data during an operation.

The endoscopic camera device 23, light source device 24, and image processing device 25 are connected to a relay unit 28 mounted on the second cart 12 via communication lines (not shown). Also, the relay unit 28 is connected to the system controller 22 mounted on the above described first cart 11 by a relay cable 29.

Thus, the system controller 22 is arranged to perform centralized control of the endoscopic camera device 23, light source device 24, and image processing device 25 which are mounted on the second cart 12, and of the electrocautery device 13, pneumoperitoneum device 14, camera device 15, light source device 16, and VTR 17 which are mounted on the first cart 11. Thus, when communication is performed between the system controllers 22 and these devices, the system controller 22 is able to display a setting state of the connected devices and a setting screen of operation switches and the like, on the above described liquid crystal display of the operation panel 21. Further, the system controller 22 is arranged to be able to perform an operation input, such as to change a set value, by allowing a desired operation switch of the operation panel 21 to be touched, and the touch panel in a predetermined region to be operated.

Further, the system controller 22 manages the operation information and control information on the endoscopic camera device 23, light source device 24, and image processing device 25, which are mounted on the second cart 12, via the relay unit 28 in real time, and captures the endoscopic image obtained by the endoscopic camera device 23.

A remote controller 30 which is a second centralized operation device operated by an operating doctor or the like located in a sterilized area, is arranged to be able to operate the other devices via the system controller 22, in the state where communication between the other devices and the system controller 22 is established.

The system controller 22 is connected to a biomedical information collection device 101 which collects biomedical information (for example, blood oxygen saturation, blood pressure, pulse and the like) from a biomedical information detecting device 100 that detects the biomedical information of the patient on the patient bed 10, via a cable 9a. The system controller 22 is capable of analyzing the biomedical information acquired from the biomedical information collection device 101, and of displaying analysis results on a required display device.

Further, the system controller 22 stores various kinds of medical information during an operation (operation information and control information of the electrocautery device 13, pneumoperitoneum device 14, endoscopic camera device 15, light source device 16, and VTR 17 which are mounted on the first cart 11, an endoscopic image obtained by the endoscopic camera device 15, operation information and control information of the endoscopic camera device 23, light source device 24, and image processing device 25 which are mounted on the second cart 12, an endoscopic image obtained by the endoscopic camera device 23, and biomedical information of the patient from the biomedical information collection device 101), and is connected to an operation information analysis device 102 which analyzes medical procedures by using these various kinds of medical information after the operation, via a cable 9b.

As shown in FIG. 2, the operation information analysis device 102 includes a medical information input section 110 as an information input section which inputs the above described various kinds of medical information during an operation from the system controller 22 via the cable 9b; a data storage section 112 as an information storage section constituted by, for example, an HDD (hard disk drive) and the like, which adds time information such as a time stamp to the various kinds of medical information during the operation and makes the time stamped medical information into files for each kind of medical information, so as to construct and store databases specified for each medical procedure; a data analysis section 114 as an information analysis section which analyzes the medical information for each. medical procedure, which medical information is formed into the databases and stored in the data storage section 112; an analysis condition input section 113 as a comparison information input section which inputs various kinds of information (for example, analysis condition and the like) to the data analysis section 114; and a display device 115 which displays analysis results analyzed by the data analysis section 114.

Note that the plural medical information files stored in the data storage section 112 are linked for each medical procedure, and are arranged to make it possible to secure the time synchronization among respective data at the time of analysis, on the basis of the time information such as a time stamp.

Further, the analysis condition input section 113 may be constituted by a pointing device, such as a keyboard and mouse. However, the analysis condition input section 113 and the display device 115 may also be constituted by a touch panel.

An effect of the present embodiment constituted as described above is explained with reference to the flow chart shown in FIG. 3, and to FIG. 4 to FIG. 11.

When an operation is started, the operation information and control information on each kind of medical instruments, image information, and further medical information including each biomedical information during the operation are managed by the system controller 22. Further, the system controller 22 outputs these kinds of medical information to the operation information analysis device 102 via the cable 9b.

The operation information analysis device 102 receives the various kinds of medical information from the system controller 22 through the medical information input section 110, and adds time information such as a time stamp to the various medical information during the operation. Then, the operation information analysis device 102 makes the various medical information to which the time information is added into files specified for each medical information, as information to be analyzed, and constructs databases specified for each medical procedure so as to store the databases in the data storage section 112. As a result, all the medical information during the operation are stored in the data storage section 112 in the state of being managed by time.

Further, in the data storage section 112, the data management for each medical procedure is performed on the basis of medical procedure ID codes. The medical procedure ID codes are inputted into the system controller 22 prior to the start of the operation, and are added to, for example, the header section of each medical information.

Thus, when the medical procedures are judged to be finished so that the medical information on the medical procedures is made into databases and stored in the data storage section 112, medical procedure analysis based on the medical information is enabled to be performed in the operation information analysis device 102.

In the analysis processing in the operation information analysis device 102, as shown in FIG. 3, first, a medical procedure ID code is inputted by the analysis condition input section 113 in step S1, and a medical procedure to be analyzed is specified.

When the medical procedure is specified, the data analysis section 114 displays an analysis item selection window on the display device 115 in step S2.

As shown in FIG. 4, this analysis item selection window 150 is a window for selecting plural items (for example, three items) to be analyzed from the items of medical information during the operation, and for improving the efficiency in the analysis by such selection.

Note that FIG. 4 shows an example in which abdominal cavity pressure data of a pneumoperitoneum device, blood oxygen saturation data, and blood pressure data are selected in the analysis item selection window 150.

Then, when the check boxes of desired items are checked on the analysis item selection window 150 by the analysis condition input section 113 and an OK button 151 is operated in step S3, the data analysis section 114 displays an analysis window 201 as shown in FIG. 5 on the display device 115 in step S4.

Note that by operating a cancel button 152, the process can be made to return to step S1 in which the medical procedure is specified.

The analysis window 201 is a window which graphically displays changing states of the information on selected items in time series and in a time-synchronized state. The analysis window 201 includes an analysis start button 202, data download button 203, threshold value setting button 204, cancel button 205 and the like, and is arranged to enable each of the buttons to be operated by a pointer 206.

When the data download button 203 is operated by the pointer 206 on the analysis window 201 in step S5, the changing states of all the data of selected items (abdominal cavity pressure data, blood oxygen saturation data, blood pressure data in FIG. 5) during the operation are graphically displayed.

Next, when the threshold value setting button 204 is operated by the pointer 206 on the analysis window 201 in step S6, threshold value lines 210 are displayed on the graphs of selected items, as shown in FIG. 6. The level of the threshold value lines 210 can be easily changed by the pointer 206. That is, the threshold value line is displayed on the basis of threshold value information for extracting a predetermined change for each information to be analyzed, and the threshold value can be changed by moving the position of the threshold value line by using the pointer 206.

Then, when the levels of the threshold value lines 210 are determined and the analysis start button 202 is operated by the pointer 206 on the analysis window 201 in step S7, markers 220 are attached as shown in FIG. 7 at the time when the values of selected items cross the threshold values. In addition, when a medically important event which is set beforehand occurs, an event occurrence area 221 which shows the occurrence period of the event is displayed.

Note that as shown in FIG. 7, an enlarged display button 207 is displayed on the analysis window 201 at this time, instead of the data download button 203 and the threshold value setting button 204.

In the example shown in FIG. 7, the data analysis section 114 extracts the period when the abdominal cavity pressure data exceeds the threshold value, that is, the concentration of carbon dioxide in the body cavity is increased, and the blood oxygen saturation becomes lower than the threshold value, as an analysis event period. Since such period is set beforehand as a medically important event, the data analysis section 114 displays this period as the event occurrence area 221, as shown in FIG. 7.

The present embodiment is arranged such that an enlarged display (time span change display) of the vicinity of event occurrence area 221 can be performed in accordance with the generation of the event occurrence area 221. Thus, the data analysis section 114 judges whether or not the enlarged display button 207 is operated by the pointer 206 in step S8. When the enlarged display button 207 is operated, predetermined instruction information is detected, so that the process proceeds to step S9. When the enlarged display button 207 is not operated, the process proceeds to step S12.

When the enlarged display button 207 is operated, the data analysis section 114 changes in step S9, as shown in FIG. 8, the time span of the graphs of items for which the event occurrence area 221 is generated, and changes the display on the screen so that the display of the time span can be enlarged in the time axis direction. At this time, thumbnails of endoscopic images at positions of the front and rear markers 220 between which the event occurrence area 221 is included, are displayed. Then, whether or not a thumbnail is selected by the pointer 206 is judged in step S10.

When a thumbnail is operated by the pointer 206, the endoscopic image during Δt period before and after the thumbnail is displayed as a moving image in the moving image reproduction window (not shown) in step S11.

Therefore, it is possible for an operator to visually analyze the state during the operation by confirming the state before and after the event occurrence area 221 with the endoscopic image. Note that the period Δt can be arbitrarily changed.

Then, when the analysis is continued in step S12, the process returns to step S2, and the processing is repeated until the stop of analysis is instructed.

FIG. 9 shows an example in which the abdominal cavity pressure data of the pneumoperitoneum device, blood oxygen saturation data, and VTR data are selected in the analysis item selection window 150. Further, FIG. 10 shows the analysis window 201 of the abdominal cavity pressure data, blood oxygen saturation data, and VTR data based on the selected items shown in FIG. 9.

For example, as shown in FIG. 10, even when the VTR recording is not performed in the event occurrence area 221, the endoscopic image is recorded as shown in FIG. 11 in the data storage section 112 constituted by the HDD. This makes it possible not only to reproduce the endoscopic image, but also to read out the moving image missing in the VTR record from the data storage section 112 and to make the read moving image recorded and stored in a DVD (not shown) or the like.

As described above, according to the present embodiment, it is possible that the medical information during the operation is classified for each medical procedure and made into databases so as to be synchronized with time, and that temporal transitions of the medical information are analyzed by selecting desired items of the medical information after the operation.

Further, it is also possible that temporal transitions of medical information are analyzed on the basis of the threshold value level for each kind of the medical information, and that a medically important event occurring over plural items is also automatically extracted.

Further, it is also possible that image information effective at the time of analysis is confirmed as a moving picture in synchronization with the above described temporal transitions of medical information.

According to the present embodiment, it is possible to obtain an effect that the medical procedures can be objectively analyzed in a simple and appropriate manner after the operation.

### Second Embodiment

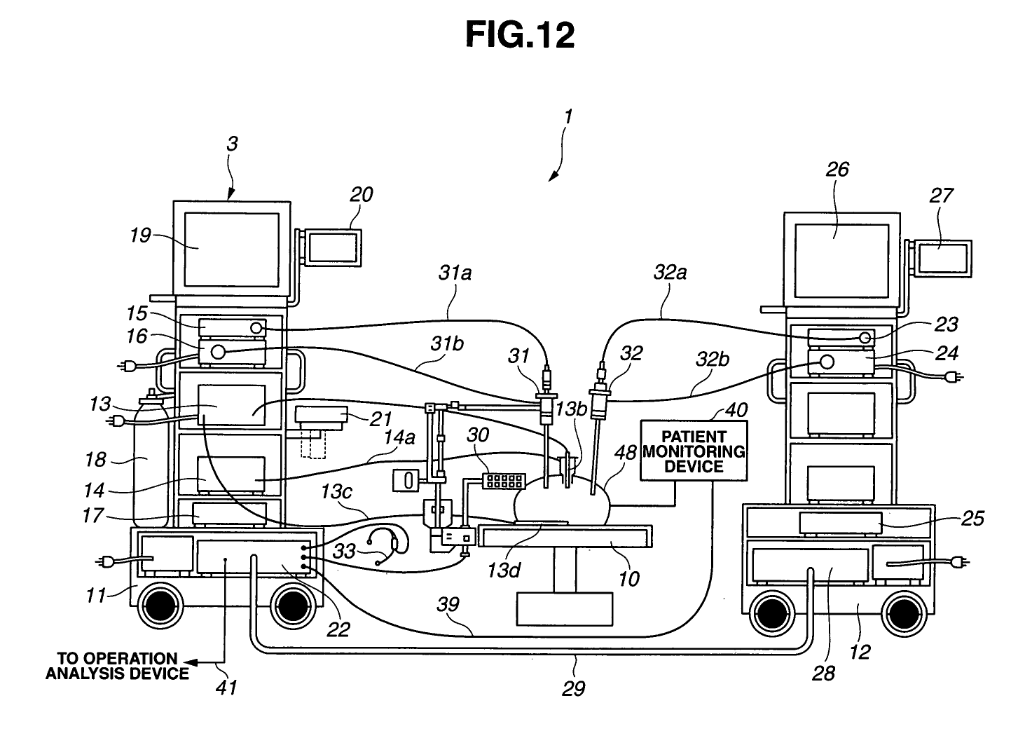

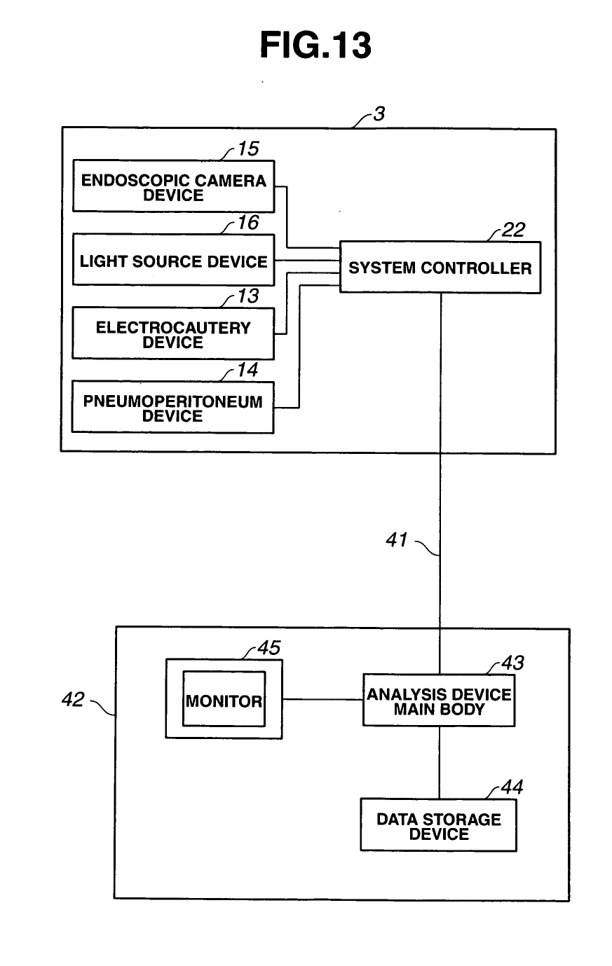

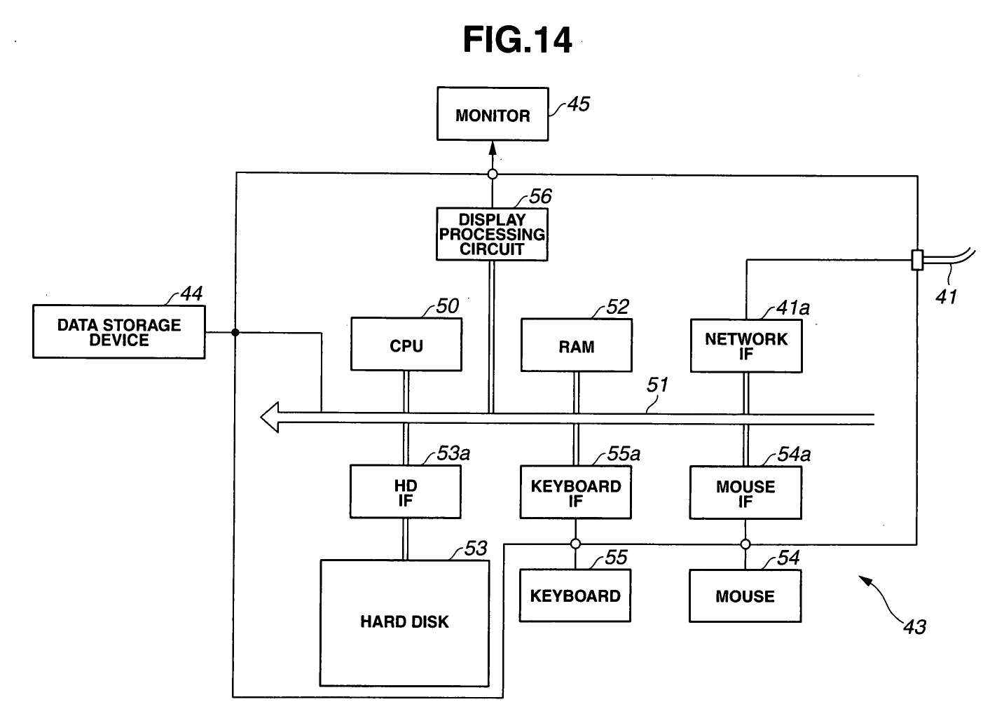

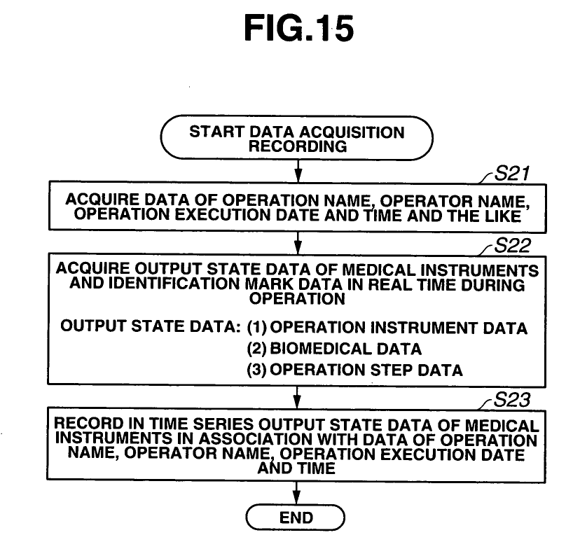

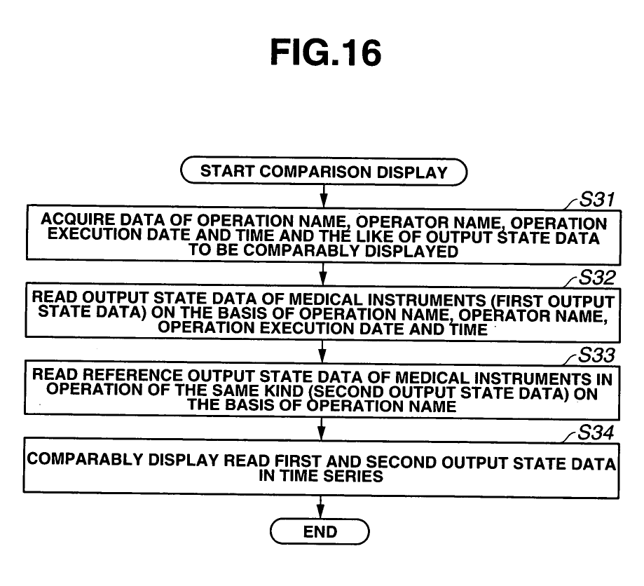

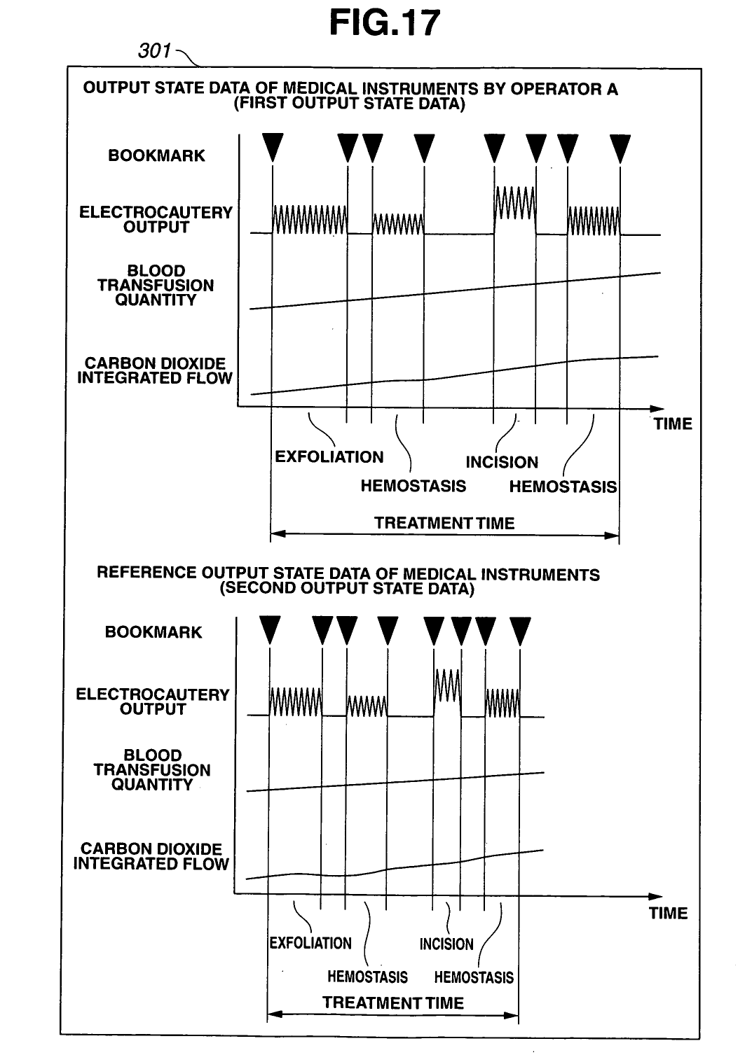

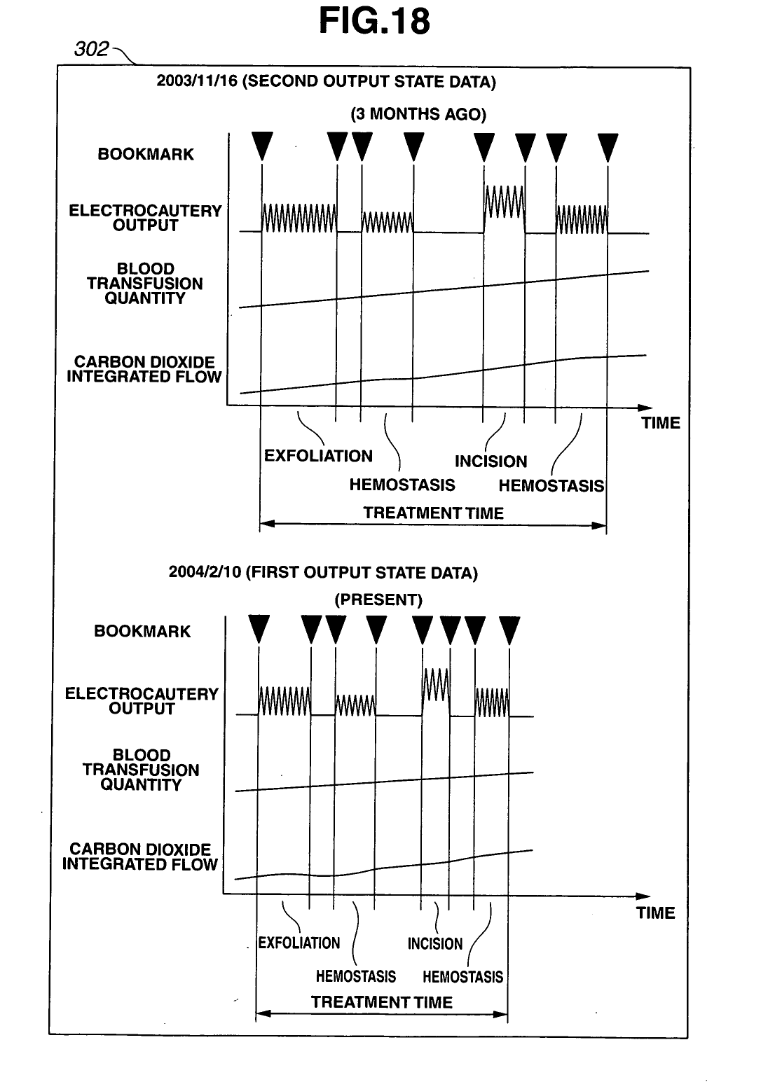

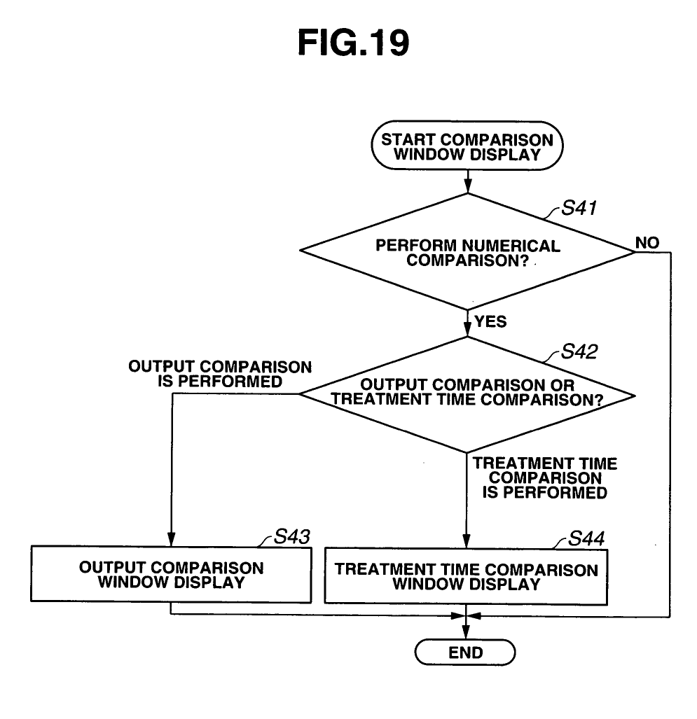

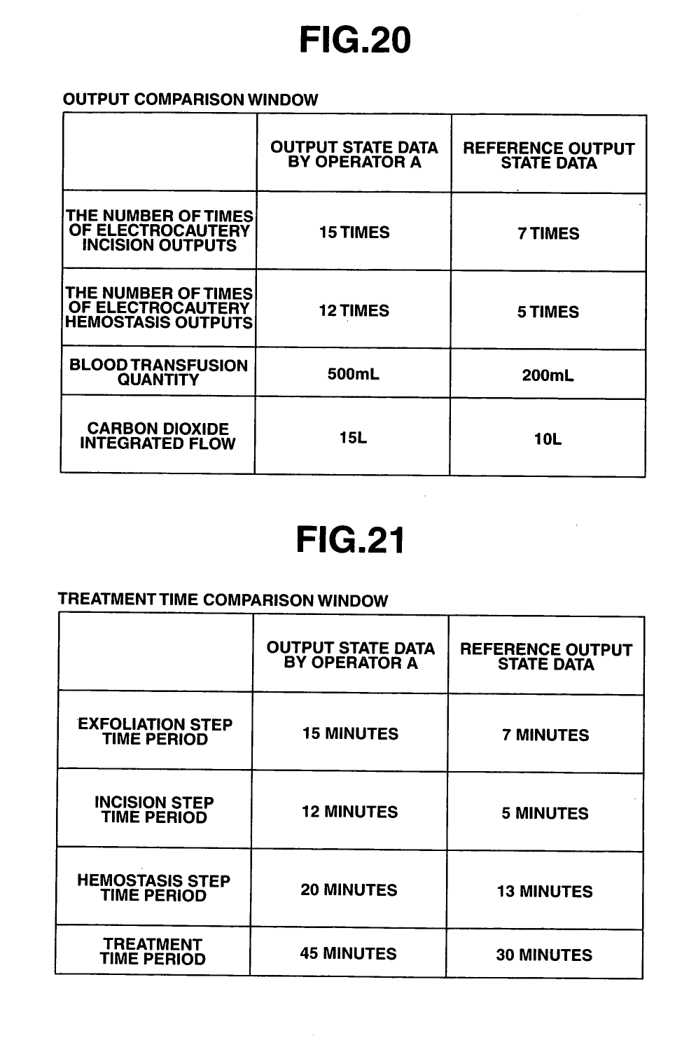

FIG. 12 to FIG. 21 are figures relating to a second embodiment according to the present invention. In the figures, FIG. 12 is a figure showing an entire constitution of an endoscopic operation system according to the second embodiment. FIG. 13 is a figure explaining an operation analysis device connected to the endoscopic operation system in FIG. 12. FIG. 14 is a block diagram showing a circuit constitution of the operation analysis device in FIG. 13. FIG. 15 is a flow chart showing a data acquisition recording operation performed by a CPU of the operation analysis device in FIG. 14. FIG. 16 is a flow chart showing comparison display processing performed by the CPU of the operation analysis device in FIG. 14. FIG. 17 is a figure showing a first comparison image which shows an example of comparison analysis with reference output state data, displayed on the monitor in FIG. 14. FIG. 18 is a figure showing a second comparison image which shows an example of comparison analysis of the skill of a same operator, displayed on the monitor in FIG. 14. FIG. 19 is a flow chart showing comparison window display processing performed by the CPU of the operation analysis device in FIG. 14. FIG. 20 is a figure showing an output comparison window which is an example of the comparison window displayed on the monitor in FIG. 14. FIG. 21 is a figure showing a treatment time comparison window which is an example of the comparison window displayed on the monitor in FIG. 14. Note that the same components as those in the first embodiment are denoted by the same reference numerals and characters, and their explanation is omitted.

As shown in FIG. 12, a patient bed 10 on which a patient 48 lies, and an endoscopic operation system 3 are arranged in an operating room 1. In FIG. 12, the same components as those in FIG. 1 are denoted by the same reference numerals and characters.

An electrocautery device 13 is arranged to perform cauterization treatment, such as incision and coagulation, to the organism tissue by applying high frequency current to the organism tissue. The electrocautery device 13 is connected to an electrocautery 13b via an active cord 13a. The electrocautery device 13 is connected with a patient plate 13d which is in contact with the buttocks of the patient in a large area, via a return cord 13c. The electrocautery device 13 is capable of performing cauterization treatment to the organism tissue in contact with the electrocautery 13b, by applying high frequency current to the organism tissue from the electrocautery 13b and by collecting the high frequency current from the patient plate 13d.

A pneumoperitoneum device 14 is connected with a carbon dioxide gas cylinder 18, so as to enable carbon dioxide gas to be supplied to the inside of the abdominal cavity of the patient via a pneumoperitoneum tube 14a. Further, the pneumoperitoneum device 14 has a function to suck the gas filled in the abdominal part and to discharge the gas to the outside.

Further, an infrared communication port (not shown) which is a communication section is attached to a system controller 22. This infrared communication port is provided in a position such as the vicinity of a display device 19, where the infrared radiation is easily irradiated, and is connected with the system controllers 22 by a cable. The system controller 22 is connected to a patient monitoring device 40 by a cable 39, and is capable of acquiring biomedical data from the patient monitoring device 40 and making the acquired data displayed on the required display device 19.

Further, the system controller 22 is connected with an operation analysis device 42 shown in FIG. 13 via a communication cable 41. Note that this operation analysis device 42 is installed, for example, in a conference room. The system controller 22 outputs identification mark data to the operation analysis device 42, in conjunction with biomedical data of the patient and operation instrument data such as of the electrocautery device. These data are recorded in the operation analysis device 42.

In the endoscopic operation system 3 according to the above described constitution, the system controller 22 acquires output state data of each kind of medical instruments in time series from the start to the end of an operation, and outputs the output state data to the operation analysis device 42. That is, the system controller 22 acquires biomedical data of the patient inputted from the patient monitoring device 40, and similarly inputted operation instrument data of instruments related to the operation, as output state data after the start of the operation, and outputs the acquired data to the operation analysis device 42 at any time. The output state data is associated with predetermined time data, such as elapsed time from the start of the operation, and is recorded in the operation analysis device 42. Specifically, the data of pulse, blood pressure, and the like, are recorded as biomedical data in the operation analysis device 42 along with time data, and further, data such as of the number of times of electrocautery output of the electrocautery device 13 are also recorded as data of instruments relating to the operation in the operation analysis device 42.

Note that the time data may be data indicating the elapsed time from the start time of the operation, which elapsed time is set to zero at the start time, or may be data of the so-called standard time in Japan which represent the real date and time. The identification mark data according to the present embodiment are also recorded in the operation analysis device 42 via the system controller 22, along with the record of the output state data.

Note that the identification mark data are arranged to be able to be inputted from a microphone 33 or endoscope switches (not shown) which are provided for first and second endoscopes 31 and 32. That is, the microphone 33 or the endoscope switch constitutes a bookmark input section.

As shown in FIG. 13 and FIG. 14, the operation analysis device 42 is constituted by having an analysis device main body 43, data storage device 44 and monitor 45.

In the analysis device main body 43, a CPU 50 which performs overall control is connected to an internal bus 51. The internal bus 51 is connected with a RAM 52 used as a work area and the like by the CPU 50, a hard disk interface (HDI/F) 53a to which a hard disk 53 storing a program, image data and the like is connected, a network interface (network I/F) 41a to which the communication cable 41 is connected, a mouse interface (mouse I/F) 54a to which a mouse 54 is connected, a keyboard interface (keyboard I/F) 55a to which a keyboard 55 is connected. Note that the mouse 54 and the keyboard 55 constitute an instruction section. Further, the internal bus 51 is connected with the monitor 45 via a display processing circuit 56 which performs display processing.

First, the CPU 50 reads the program stored in the hard disk 53, and writes the program in a predetermined region in the RAM 52, and thereafter operates in accordance with the program. The CPU 50 reads the operation instrument data and biomedical data which are stored in the data storage device 44, so as to make the read data stored in the hard disk 53. Then, the CPU 50 analyzes the data and performs editing processing such as display processing. Therefore, the data storage device 44 is provided with a function as data recording section which records plural kinds of output state data acquired during the operation.

For example, it is possible for an operating doctor (operator) in charge of the operation in the operating room 1, to input a command into the CPU 50 by operating the keyboard 55 after the operation, and to fetch plural kinds of operation instrument data of the operation instruments, such as the electrocautery device 13, pneumoperitoneum device 14, and ultrasonic treatment device (not shown), which data are recorded in the data storage device 44, and the output state data such as of the biomedical data from the patient monitoring device 40 into the hard disk 53 via the network I/F 41a.

Thereby, the operator is enabled to edit the output state data and the like stored in the hard disk 53, so as to create a file suitable to be used in the diagnosis and succeeding operation or the like, and is also enabled to read and utilize the desired other data or the like from other server (not shown) or the like, as needed.

According to the present embodiment, in the data analysis after the operation, and the like, it is possible to display the output state data of medical instruments in a manner that plural kinds of output state data are displayed on the same time base. Further, in the present embodiment, it is possible to make a comparison with the reference output state data of the medical instruments or with the output state data of preceding operations, by simultaneously displaying the reference output state data of the medical instruments or the output state data of preceding operations.

That is, the CPU 50 controls the display processing circuit 56, so as to make graphical display processing for displaying the operation instrument data and biomedical data on the same time base performed, and to make the output state data of medical instruments displayed on the monitor 45. Further, the CPU 50 reads out the reference output state data of medical instruments recorded beforehand in the data storage device 44, or the output state data of preceding operations. Thereby, the CPU 50 controls the display processing circuit 56, so as to make the reference output state data of medical instruments or the output state data of preceding operations simultaneously displayed.

The output state data of medical instruments are influenced by the body shape, such as corpulent type and skinny type, and the constitution of the patient. However, the reference output state data are reference values which are set as a target by the operator, and are calculated from a standard model based on the patient of a standard body shape.

When the endoscopic operation by means of the endoscopic operation system 3 in the operating room 1 is started, the operation analysis device 42 constituted in this way acquires the output state data, as described above.

First, the endoscopic operation system 3, as shown in FIG. 12, is constituted in the operating room 1 by an operator and the like.

An operator, such as a nurse who is located in the non-sterilized area, operates an operation panel 21, and inputs data such as an operation name, operator name, and operation execution date and time. The inputted data are transmitted to the system controller 22. The operator wears the headset type microphone 33. and starts the operation. At this time, the operator voices “operation started” to the microphone 33.

The system controller 22 starts counting in a timer (not shown) on the basis of the voice signal of “operation started” from the microphone 33, to start the measuring of time. The system controller 22 outputs the data of “operation started” and the data of start time to the operation analysis device 42.

At the same time, the system controller 22 starts to acquire the operation instrument data, biomedical data, and operation step data as output state data of connected medical instruments, and identification mark data.

More specifically, the system controller 22 acquires, for example, operation instrument data such as of electrocautery output data outputted from the electrocautery device 13, biomedical data, such as blood pressure, pulse, blood transfusion quantity of the patient, outputted from the patient monitoring device 40, and the like.

Further, the system controller 22 acquires operation step data. The operation step data are, for example, “incision” operation data and the like which are operation data outputted from the electrocautery device 13, when incision treatment by the electrocautery device 13 is performed.

While the endoscopic operation is performed, the operator inputs identification mark data so as to make the identification mark data serve as reference points in the operation analysis after the operation. The identification mark data are inputted by the voice of the operator with the microphone 33 or by endoscope switches provided for the first and second endoscopes 31 and 32. The inputted identification mark data are inputted to the system controller 22.

The system controller 22 transmits the acquired data such as the operation name, operator name, and operation execution date and time, the output state data of medical instruments (operation instrument data, biomedical data and operation step data), and the identification mark data to the operation analysis device 42 provided in the conference room via the communication cable 41.

The operation analysis device 42 receives and records the data transmitted from the system controller 22. The data acquisition recording operation of the operation analysis device 42 is performed under the control of the CPU 50 in the analysis device main body 43 in accordance with the flow chart shown in FIG. 15.

As shown in FIG. 15, the CPU 50 acquires the data of operation name, operator name, operation execution date and time and the like (step S21). Next, the CPU 50 acquires the output state data of medical instruments in real time during the operation (step S22). The output state data of medical instruments are the operation instrument data, biomedical data, and operation step data, as described above.

Next, the CPU 50 makes the acquired output state data of medical instruments linked to data such as the operation name, operator name, and operation execution date and time, to thereby record the linked output state data in time series in the data storage device 44 (step S23). At this time, the CPU 50 makes the output state data (operation instrument data, biomedical data and operation step data) from medical instruments associated with the identification mark data, to thereby record the associated output state data in the data storage device 44.

Accordingly, the operation analysis device 42 is enabled to receive and record the data transmitted from the system controller 22.

When finishing the operation, the operator voices “operation finished” to the microphone 33. The system controller 22 stops the counting in the timer on the basis of the voice signal of “operation finished” from the microphone 33. At the same time, the system controller 22 stops acquiring the output state data of connected medical instruments.

Further, the system controller 22 outputs the data of “operation finished” and the data of operation finish time to the operation analysis device 42. Note that in the operation analysis device 42, the data of “operation started”, operation start time data, data of “operation finished”, and operation finish time data are also recorded as the data of operation execution date and time in the data storage device 44.

After the above described endoscopic operation, the operator enters the conference room, and reads and analyzes the output state data by operating the operation analysis device 42, so as to prepare a medical record or the like, and also to prepare a scientific essay to be presented at a meeting or the like. At this time, the operator verifies the output state data of medical instruments during the executed operation, by comparison with the reference output state data. The operator operates the operation analysis device 42 to make the output state data of medical instruments and the reference output state data compared and displayed.

The comparison display processing by the operation analysis device 42 is performed under the control of the CPU 50 of the analysis device main body 43 in accordance with the flow chart shown in FIG. 16. First, by using the mouse 54 or keyboard 55 of the operation analysis device 42, the operator inputs the data of operation name, operator name, operation date and time, and the like, corresponding to the output state data to be compared and displayed, and makes the comparison and display performed.

As shown in FIG. 16, the CPU 50 acquires the data of operation name, operator name, operation execution date and time or the like, which are to be compared and displayed, on the basis of the input of the operator (step S31). Next, the CPU 50 reads the output state data (first output state data) of medical instruments recorded in the data storage device 44 on the basis of the acquired data of operation name, operator name, operation execution date and time or the like (step S32). At this time, the CPU 50 stores the output state data (first output state data) of medical instruments read from the data storage device 44, in the hard disk 53.

Next, the CPU 50 reads the output state data (second output state data) of medical instruments in an operation of the same kind, which data are recorded beforehand in the data storage device 44, on the basis of the acquired operation name (step S33). At this time, the CPU 50 stores the output state data (second output state data) of medical instruments read from the data storage device 44, in the hard disk 53.

The CPU 50 performs processing to display comparison between the read first and second output state data in time series (step S34). The CPU 50 controls the display processing circuit 56, so as to make the first and second output state data stored in the hard disk 53 read at any time, and to make graphic display processing performed.

The display processing circuit 56 performs the graphic display processing on the basis of the first and second output state data, and displays image data obtained by performing the graphic display processing on the monitor 45. At this time, the display processing circuit 56 performs the graphic display processing for displaying on the same time base, the operation instrument data and biomedical data that are associated with the identification mark data, and displays the output state data of medical instruments. The first and second output state data subjected to the graphic display processing as described above, are displayed as a comparison image on the monitor 45, for example, as shown in FIG. 17.

The comparison image shown in FIG. 17 is an example of a comparison analysis with the reference output state data.

In the comparison image 301, the output state data of medical instruments used by the operator A are displayed as the first output state data on the upper stage, the reference output state data are displayed as the second output state on the lower stage. In these graphs, for example, the electrocautery output data as the operation instrument data, and the blood transfusion quantity and carbon dioxide integrated flow data as the biomedical data are displayed on the same time base in association with the identification mark data.

Further, the operation data outputted from operation instruments, that is, the operation data such as “exfoliation”, “hemostasis” and “incision” which are the operation data outputted from the electrocautery device 13 in the figure, are displayed in these graphs. Further, the treatment time (operation time) is displayed under the display of the operation data in these graphs.

In the present embodiment, the identification mark data are inputted in accordance with the operation (treatment) of the electrocautery device 13, and relationships of the treatment operation of the electrocautery device 13 which is performed during the periods between the identification mark data, to the blood transfusion quantity and the carbon dioxide integrated flow which are detected by the patient monitoring device 40, are displayed as the output state data of medical devices.

The operator is enabled to verify the output state data of medical devices in the executed operation by comparison with the reference output state data, by making reference to the comparison image 301 with such reference output state data, and to perform objective analysis of the operation.

Therefore, the operation analysis device 42 facilitates the comparison with the reference output state data of medical instruments, and makes it possible to objectively evaluate the skill or the like of the operator who has performed the operation. Note that the comparison image is an example of comparison with the reference output state data of medical instruments, but of course, a display of comparison with the output state data of preceding operations performed by an operator different from the operator A, or a display of comparison with the output state data of a skilled operator as a target is also possible.

Further, as the comparison image 301, for example, as shown in FIG. 18, the output state data of a preceding operation of the same kind performed at different date and time by the same operator, may also be simultaneously displayed in order to analyze the skill of the operator.

The comparison image 302 shown in FIG. 18 is an example of comparison analysis of the skill of the same operator.

In the comparison image 302, the output state data in the operation performed by the operator A three months ago are graphically displayed as the second output state data on the upper stage, while the output state data in the present operation performed by the operator A are graphically displayed as the first output state data on the lower stage. Note that these graphs are constituted similarly to those displayed in the comparison image shown in FIG. 17, and hence, their explanation is omitted.

Accordingly, the operator is enabled to verify the output state data of medical devices in the operation performed at this time by comparison with the output state data at the different date and time, by making reference to such skill comparison image 302, and to thereby objectively analyze the skill of the operation performed by the operator oneself.

Therefore, the operation analysis device 42 facilitates the comparison with the output state data of medical instruments in the preceding operations of the same kind, so as to enable the operator to objectively evaluate the skill on the operation performed by the operator oneself. Note that in the present embodiment, the output state data of medical instruments are configured to be displayed by one graph on the same time base in association with the identification mark data, but the output state data of medical instruments may also be displayed by individual graphs or plural graphs on the same time base in association with the identification mark data.

Further, when performing the comparison and verification on the basis of the comparison image, the operator may desire to make the output state data of each medical instrument numerically represented. At this time, the operator operates the mouse 54 or the keyboard 55 of the operation analysis device 42, so as to make the comparison window in which the output state data of each medical instrument are numerically represented, displayed. The comparison window display processing in the operation analysis device 42 is performed under the control of the CPU 50 of the analysis device main body 43 in accordance with the flow chart shown in FIG. 19.

As shown in FIG. 19, the CPU 50 judges whether or not the numerical comparison is performed, on the basis of the input by the operator (step S41). When the numerical comparison is not performed, the CPU 50 ends the comparison window display processing. When the numerical comparison is performed, the CPU 50 judges which of the medical instrument output comparison and the treatment time comparison is performed (step S42).

When performing the output comparison of medical instruments, the CPU 50 displays the output comparison window of medical instruments (step S43). In this case, the CPU 50 generates the output comparison window data of medical instruments, on the basis of the first and second output state data which are compared and displayed in the comparison image, and controls the display processing circuit 56 so as to make the output comparison window data displayed on the monitor 45.

On the other hand, when performing the treatment time comparison, the CPU 50 displays the treatment time comparison window of medical instrument (step S44). In this case, the CPU 50 generates the treatment time comparison window data of medical instruments on the basis of the first and second output state data which are compared and displayed in the comparison image, and controls the display processing circuit 56 so as to make the treatment time comparison window data displayed on the monitor 45.

In this way, the output comparison window of medical instruments which are subjected to the comparison window display processing is displayed on the monitor 45 as shown in FIG. 20, and the treatment time comparison window is displayed on the monitor 45 as shown in FIG. 21.

In the output comparison window of medical instruments shown in FIG. 20, for example, data of the number of times of electrocautery incision outputs, data of the number of times of electrocautery hemostasis outputs, as the operation instrument data, and blood transfusion quantity data and carbon dioxide integrated flow data which are the biomedical data, are numerically represented.

Further, in the treatment time period comparison window of medical instruments shown in FIG. 21, for example, the time periods of exfoliation step, incision step, hemostasis step, which steps are performed by the electrocautery device 13 as an operation instrument, and the whole treatment time period data are numerically represented.

Thus, by making reference to such comparison window displays, the operator is able to verify the output state data of medical devices used in the executed operation by numerical comparison with the reference output state data, and to further objectively analyze the operation. Note that the comparison window display is an example of comparison with the reference output state data of medical instruments, similar to the above described comparison image, and it is of course possible to display a numerical comparison with the output state data of preceding operations performed by an operator different from the operator A or with the output state data of a skilled operator to be targeted.

As a result, the operation analysis device 42 is capable of displaying on the same time base, the output state data of medical instruments which are recorded in association with the identification mark data. Thus, the operation analysis device 42 is capable of making the data easily associated with each other, and thereby making the contents of the operation and the state during the operation easily confirmed.

Therefore, the operation analysis device 42 makes it easier to confirm the output state data of medical instruments in time series, and to compare the output state data of medical instruments with the reference output state data of medical instruments or the output state data of preceding operations, thereby making it possible to objectively evaluate the skill of the operator who has performed the operation. Further, when a medical instrument such as the electrocautery device 13 is replaced with a latest medical instrument, the operation analysis device 42 is capable of analyzing the time shortening effect based on the instrument change by performing the comparison with the use profile of the medical instrument during the periods close to each other.

Note that in the present embodiment, the operation analysis device 42 is constituted by applying the present invention to the endoscopic operation system 3. However, the present invention is not limited to this constitution, and the operation analysis device 42 may also be constituted by applying the present invention to an operation system for performing an abdominal operation.

Further, the operation analysis device according to the present embodiment makes it easier to confirm the output state data of medical instruments in time series, and to compare the output state data of medical instruments with the reference output state data of medical instruments or the output state data of preceding operations, thereby making it possible to objectively evaluate the skill and the like of the operator who has performed the operation. Thus, the operation analysis device according to the present embodiment is suitable for the data analysis in preparing a medical record, scientific essay and the like.

As described above, the operation analysis device according to the present embodiment has an effect of making it easier to confirm the output state data of medical instruments in time series, and to compare the output state data of medical instruments with the reference output state data of medical instruments or the output state data of preceding operations, thereby making it possible to objectively evaluate the skill and the like of the operator who has performed the operation.

Note that an embodiment constituted such as by partially combining the above described embodiments is also included within the scope of the present invention.

The present invention is not limited to the above described embodiments, and can be practiced with various modification, changes and the like without departing from the scope of the invention.

What is claimed is:

1. An operation information analysis device comprising:
an information input section for inputting plural pieces of instrument information of plural medical instruments and plural pieces of biomedical information;
an information storage section for adding time information to the plural pieces of instrument information and the plural pieces of biomedical information, that are inputted by the information input section, and for classifying and storing the plural pieces of instrument information and the plural pieces of biomedical information in association with predetermined codes, as information to be analyzed;
a comparison information input section for inputting comparison information to be compared with the information to be analyzed; and
an information analysis section for analyzing the information to be analyzed by comparing the information to be analyzed with the comparison information.

2. The operation information analysis device according to claim 1,
wherein the comparison information is threshold value information for extracting predetermined changes for each piece of the information to be analyzed.

3. The operation information analysis device according to claim 2,
wherein when the predetermined changes occur at a same time zone based on the time information, in the previously specified plural pieces of information to be analyzed, the information analysis section extracts the time zone as an analysis event period.

4. The operation information analysis device according to claim 3,
wherein the information analysis section displays the extracted analysis event period as an event occurrence area on a screen of a display device.

5. The operation information analysis device according to claim 4,
wherein the information analysis section expands the display of the plural pieces of information to be analyzed during the analysis event period on the screen, in the time axis direction in accordance with predetermined instruction information.

6. The operation information analysis device according to claim 4,
wherein the plural pieces of instrument information include image information, and
wherein the information analysis section extracts the image information at the occurrence time of the predetermined changes on the basis of the time information, and displays the extracted image information as a thumbnail image on the screen.

7. The operation information analysis device according to claim 5,
wherein the plural pieces of instrument information include image information, and
wherein the information analysis section extracts the image information at the occurrence time of the predetermined changes on the basis of the time information, and displays the extracted image information as a thumbnail image on the screen.

8. The operation information analysis device according to claim 6,
wherein the image information is an endoscopic image captured by an endoscope.

9. The operation information analysis device according to claim 8,
wherein the predetermined codes are codes about medical procedures.

10. The operation information analysis device according to claim 7,
wherein the image information is an endoscopic image captured by an endoscope.

11. The operation information analysis device according to claim 10,
wherein the predetermined codes are codes about medical procedures.

12. An operation information analysis method comprising:
inputting plural pieces of instrument information of plural medical instruments and plural pieces of biomedical information;
adding time information to the inputted plural pieces of instrument information and the inputted plural pieces of biomedical information, and classifying and storing the plural pieces of instrument information and the plural pieces of biomedical information in association with predetermined codes, as information to be analyzed; and
analyzing the information to be analyzed by comparing the information to be analyzed with comparison information to be compared with the information to be analyzed.

13. An operation information analysis device comprising:
a data recording section for recording output state data including operation instrument data outputted from medical instruments, in association with identification mark data for identifying each treatment performed by the medical instruments, the identification mark data being inputted from a bookmark input section; and
a display processing section for comparably displaying first output state data representing the output state data and the identification mark data that are recorded in the data recording section, on a same time base, and second output state data representing other output state data and other identification mark data that are recorded beforehand in the data recording section, on the same time base.

14. The operation information analysis device according to claim 13,
wherein the display processing section is capable of displaying previously recorded reference output state data, previously recorded output state data of a different operator, or other output state data of a same operator that are recorded at different date and time, as the second output state data.

15. The operation information analysis device according to claim 14,
wherein the same time base is a time base of elapsed time from start of an operation.

16. The operation information analysis device according to claim 15,
wherein the display processing section displays the first output state data and the second output state data in graph in accordance with the same time base.

17. The operation information analysis device according to claim 16,
wherein the bookmark input section is constituted by a microphone or an endoscope switch.

18. The operation information analysis device according to claim 17,
wherein the output state data include biomedical data of a patient outputted from a patient monitoring device.

19. The operation information analysis device according to claim 18,
wherein the output state data include operation step data outputted from the medical instruments.

20. An operation information analysis method comprising:
recording output state data including operation instrument data outputted from medical instruments, in association with identification mark data for identifying each treatment performed by the medical instruments, the identification mark data being inputted from a bookmark input section; and
comparably displaying first output state data representing the output state data and the identification mark data that are recorded, on a same time base, and second output state data representing other output state data and other identification mark data that are recorded beforehand in the data recording section, on the same time base.
